(19) United States
(12) Patent Application Publication
(10) Pub. No.: US 2024/0182457 A1
(43) Pub. Date: Jun. 6, 2024

(54) OXADIAZOLE-SUBSTITUTED SPIROCYCLIC COMPOUND AND APPLICATION THEREOF

(71) Applicants: HELIOEAST PHARMACEUTICAL CO., LTD., Jiangxi (CN); HELIOEAST SCIENCE & TECHNOLOGY CO., LTD., Jiangxi (CN)

(72) Inventors: Lingyun WU, Shanghai (CN); Xu YOU, Shanghai (CN); Lele ZHAO, Shanghai (CN); Shuhui CHEN, Shanghai (CN)

(21) Appl. No.: 18/551,603

(22) PCT Filed: Apr. 2, 2022
(86) PCT No.: PCT/CN2022/085113

§ 371 Date: Sep. 21, 2023

(30) Foreign Application Priority Data
Apr. 9, 2021 (CN) 202110384689.2

(51) Int. Cl. C07D 413/04; A61K 31/4245

(52) U.S. Cl. CPC C07D 413/04; A61K 31/4245

(57) ABSTRACT

Provided is a compound as represented by formula (I) or a pharmaceutically acceptable salt thereof.

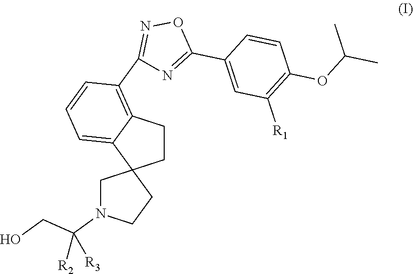

### TECHNICAL FIELD

The present disclosure relates to a series of oxadiazole-substituted spirocyclic compounds and a use thereof, specifically to a compound of formula (I) or a pharmaceutically acceptable salt thereof.

### BACKGROUND

Sphingosine-1-phosphate (S1P) is a multifunctional lipid mediator with a broad spectrum of physiological activities, including cell proliferation, cell survival, lymphocyte trafficking, cytoskeletal organization, and morphogenesis. Sphingosine is catalyzed by ceramidase and is released from ceramide. In the presence of sphingosine kinase, sphingosine undergoes phosphorylation to produce sphingosine-1-phosphate (S1P), which interacts with sphingosine-1-phosphate receptor (S1PR) to elicit physiological activities.

Sphingosine-1-phosphate receptor 1 (S1PR1), also known as endothelial differentiation gene 1 (EDG1), is a G-protein coupled receptor that belongs to the endothelial differentiation gene (EDG) receptor family. It is a protein encoded by the S1PR1 gene. The sphingosine-1-phosphate receptor (S1PR) comprises five subtypes (S1PR1 to 5), among which sphingosine-1-phosphate receptor 1 (S1PR1) is abundantly distributed on the endothelial cell membrane. Like other G-protein coupled receptors, S1PR1 detects its ligand extracellularly and activates intracellular signaling pathways, leading to a cellular response.

Sphingosine-1-phosphate (S1P) is crucial in the human body, as it primarily regulates the vascular and immune systems. Small molecule S1P1 agonists and inhibitors mimic the binding mechanism of sphingosine-1-phosphate (S1P) to its receptor and have been shown to play significant physiological roles in its signaling system. Activation of sphingosine-1-phosphate receptor 1 (S1PR1) disrupts lymphocyte trafficking, sequestering lymphocytes in lymph nodes and other secondary lymphoid organs, leading to rapid and reversible lymphopenia. Clinical studies have shown that the sequestration of lymphocytes reduces inflammatory or autoimmune responses, which is crucial for immunomodulation.

Currently, the in vivo pharmacodynamic studies of sphingosine-1-phosphate receptor 1 (S1PR1) agonists have been disclosed and intended for the treatment or prevention of autoimmune diseases. The discovery and application of novel sphingosine-1-phosphate receptor 1 (S1PR1) agonists hold great promise.

### Content of the Present Invention

The present disclosure provides a compound of formula (I) or a pharmaceutically acceptable salt thereof,

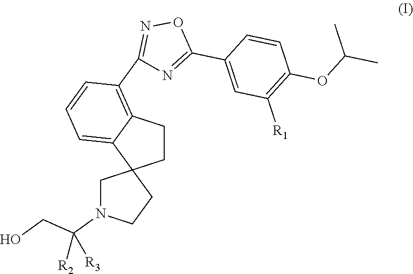

wherein
R₁ is selected from H, F, Cl, Br, I, OH, NH₂, CN, C₁₋₃ alkyl, and C₁₋₃ alkoxy, wherein the C₁₋₃ alkyl and C₁₋₃ alkoxy are each independently and optionally substituted by 1, 2, or 3Rₐ;
R₂ is selected from H, C₁₋₃ alkyl, and C₁₋₃ alkoxy, wherein the C₁₋₃ alkyl and C₁₋₃ alkoxy are each independently and optionally substituted by 1, 2, or 3 R_{b};
each R₃ is independently selected from C₁₋₃ alkyl and C₁₋₃ alkoxy, wherein the C₁₋₃ alkyl and C₁₋₃ alkoxy are each independently and optionally substituted by 1, 2, or 3 R_{c};
or, R₂ and R₃ together with the carbon atom to which they are attached form a C₃₋₇ cycloalkyl ring;
Rₐ, R_{b}, and Re are each independently selected from F, Cl, Br, I, OH, NH₂, CN, and COOH.

In some embodiments of the present disclosure, the Rₐ, R_{b}, and R_{c} are each independently selected from F, Cl, and Br, and other variables are as defined in the present disclosure.

In some embodiments of the present disclosure, the R₁ is selected from H, F, Cl, Br, CN, —CH₃, and

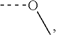

wherein the —CH₃ and —OCH₃ are each independently and optionally substituted by 1, 2, or 3 Rₐ, and Rₐ and other variables are as defined in the present disclosure.

In some embodiments of the present disclosure, each Riis independently selected from H, F, Cl, Br, CN, —CH₃,

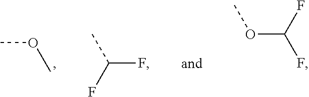

and other variables are as defined in the present disclosure.

In some embodiments of the present disclosure, the R₂ is selected from H, —CH₃, —CH₂CH₃,

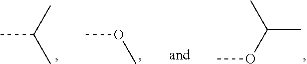

wherein the —CH₃, —CH₂CH₃,

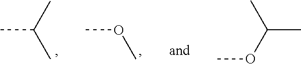

are each independently and optionally substituted by 1, 2, or 3 R_{b}, and R_{b} and other variables are as defined in the present disclosure.

In some embodiments of the present disclosure, the R₂ is selected from H, —CH₃, —CH₂CH₃,

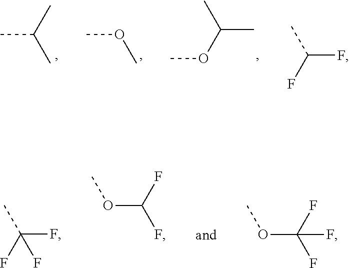

and other variables are as defined in the present disclosure.

In some embodiments of the present disclosure, the R₃ is selected from —CH₃, —CH₂CH₃,

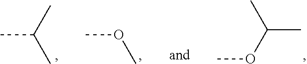

wherein the —CH₃, —CH₂CH₃,

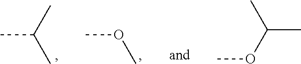

are each independently and optionally substituted by 1, 2, or 3 R_{c}, and Re and other variables are as defined in the present disclosure.

In some embodiments of the present disclosure, the R₃ is selected from —CH₃, —CH₂CH₃,

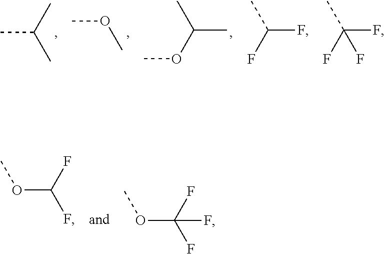

and other variables are as defined in the present disclosure.

In some embodiments of the present disclosure, the R₂ and R₃ together with the carbon atom to which they are attached form

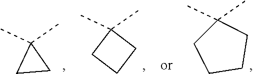

and other variables are as defined in the present disclosure.

In some embodiments of the present disclosure, the structural moiety

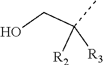

is selected from

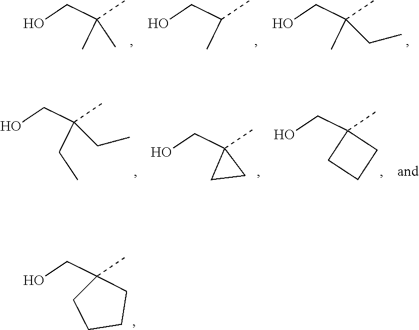

and other variables are as defined in the present disclosure.

In some embodiments of the present disclosure, the structural moiety

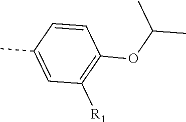

is selected from

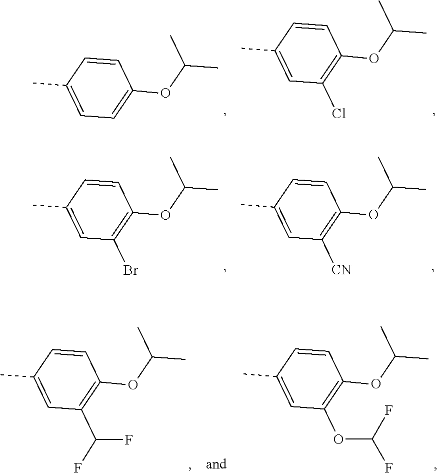

and other variables are as defined in the present disclosure.

There are still some embodiments of the present disclosure which are obtained by any combination of the above variables.

The present disclosure also provides a compound of the following formulas or a pharmaceutically acceptable salt thereof,

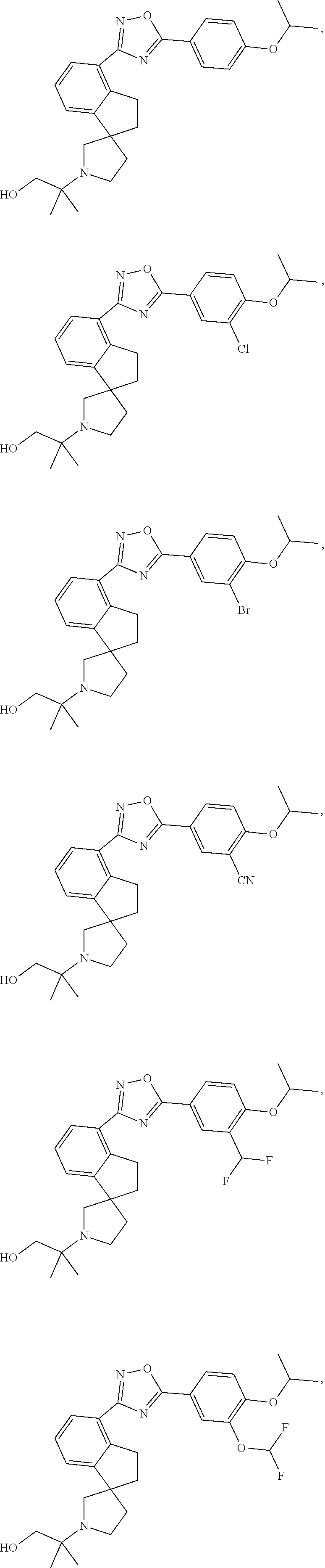 [SMILES: CC(C)Oc1ccc(-c2nc(-c3cccc4c3CCC43CCN(C(C)(C)CO)C3)no2)cc1 ; CC(C)Oc1ccc(-c2nc(-c3cccc4c3CCC43CCN(C(C)(C)CO)C3)no2)cc1Cl ; CC(C)Oc1ccc(-c2nc(-c3cccc4c3CCC43CCN(C(C)(C)CO)C3)no2)cc1Br ; CC(C)Oc1ccc(-c2nc(-c3cccc4c3CCC43CCN(C(C)(C)CO)C3)no2)cc1C#N ; CC(C)Oc1ccc(-c2nc(-c3cccc4c3CCC43CCN(C(C)(C)CO)C3)no2)cc1C(F)F ; CC(C)Oc1ccc(-c2nc(-c3cccc4c3CCC43CCN(C(C)(C)CO)C3)no2)cc1OC(F)F]

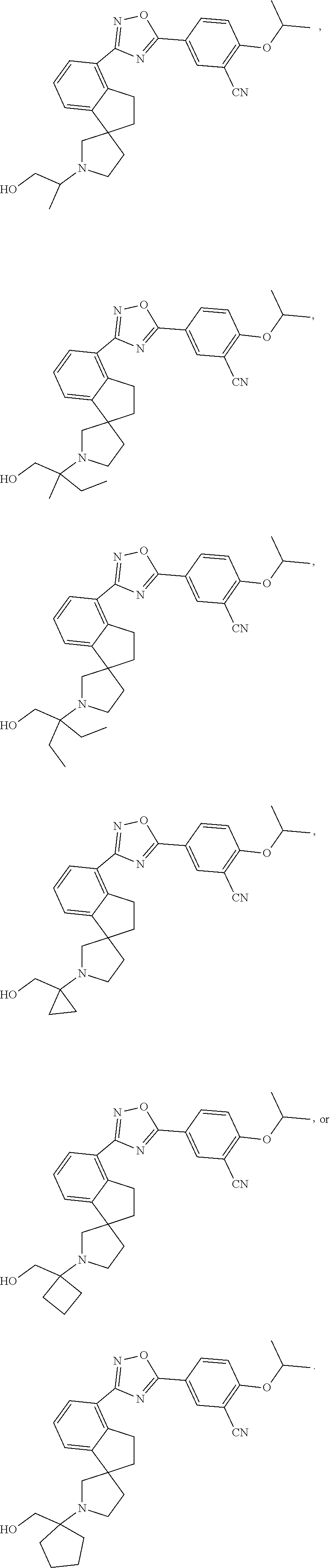 [SMILES: CC(C)Oc1ccc(-c2nc(-c3cccc4c3CCC43CCN(C(C)CO)C3)no2)cc1C#N ; CCC(C)(CO)N1CCC2(CCc3c(-c4noc(-c5ccc(OC(C)C)c(C#N)c5)n4)cccc32)C1 ; CCC(CC)(CO)N1CCC2(CCc3c(-c4noc(-c5ccc(OC(C)C)c(C#N)c5)n4)cccc32)C1 ; CC(C)Oc1ccc(-c2nc(-c3cccc4c3CCC43CCN(C4(CO)CC4)C3)no2)cc1C#N ; CC(C)Oc1ccc(-c2nc(-c3cccc4c3CCC43CCN(C4(CO)CCC4)C3)no2)cc1C#N ; CC(C)Oc1ccc(-c2nc(-c3cccc4c3CCC43CCN(C4(CO)CCCC4)C3)no2)cc1C#N]

The present disclosure also provides a compound of the following formulas or a pharmaceutically acceptable salt thereof,

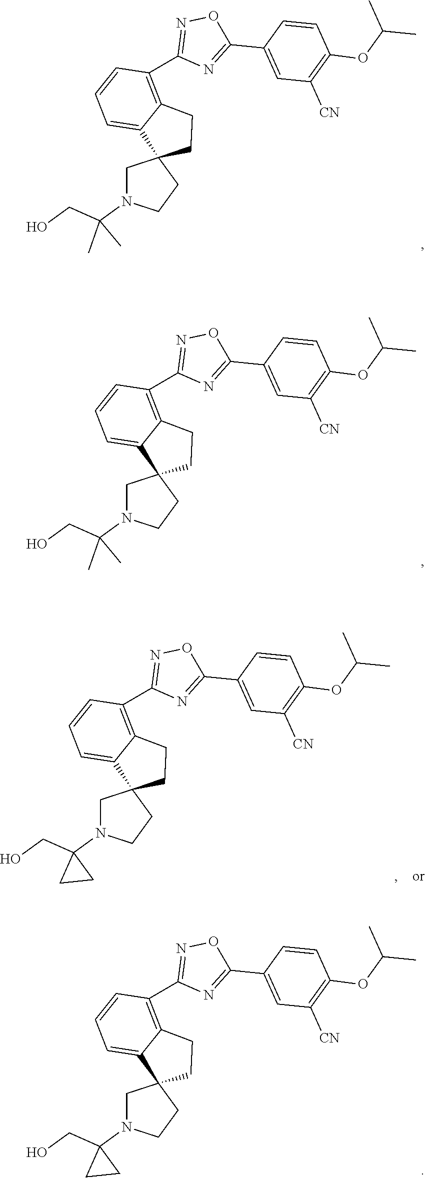 [SMILES: CC(C)Oc1ccc(-c2nc(-c3cccc4c3CC[C@]43CCN(C(C)(C)CO)C3)no2)cc1C#N ; CC(C)Oc1ccc(-c2nc(-c3cccc4c3CC[C@@]43CCN(C(C)(C)CO)C3)no2)cc1C#N ; CC(C)Oc1ccc(-c2nc(-c3cccc4c3CC[C@]43CCN(C4(CO)CC4)C3)no2)cc1C#N ; CC(C)Oc1ccc(-c2nc(-c3cccc4c3CC[C@@]43CCN(C4(CO)CC4)C3)no2)cc1C#N]

The present disclosure also provides a use of the compound or the pharmaceutically acceptable salt thereof in the manufacture of a medicament for treating S1PR1 receptor.

Technical Effect

The compound of the present disclosure exhibits significant S1PR1 agonist activity and favorable bioavailability.

Definition and Description

Unless otherwise specified, the following terms and phrases when used herein have the following meanings. A specific term or phrase should not be considered indefinite or unclear in the absence of a particular definition, but should be understood in the ordinary sense.

When a trading name appears herein, it is intended to refer to its corresponding commercial product or active ingredient thereof.

The term “pharmaceutically acceptable” is used herein in terms of those compounds, materials, compositions, and/or dosage forms, which are suitable for use in contact with human and animal tissues within the scope of reliable medical judgment, with no excessive toxicity, irritation, an allergic reaction or other problems or complications, commensurate with a reasonable benefit/risk ratio.

The term “pharmaceutically acceptable salt” refers to a salt of the compound of the present disclosure that is prepared by reacting the compound having a specific substituent of the present disclosure with a relatively non-toxic acid or base. When the compound of the present disclosure contains a relatively acidic functional group, a base addition salt can be obtained by bringing the compound into contact with a sufficient amount of base in a pure solution or a suitable inert solvent. The pharmaceutically acceptable base addition salt includes a salt of sodium, potassium, calcium, ammonium, organic amine or magnesium, or similar salts. When the compound of the present disclosure contains a relatively basic functional group, an acid addition salt can be obtained by bringing the compound into contact with a sufficient amount of acid in a pure solution or a suitable inert solvent. Examples of the pharmaceutically acceptable acid addition salt include an inorganic acid salt, wherein the inorganic acid includes, for example, hydrochloric acid, hydrobromic acid, nitric acid, carbonic acid, bicarbonate, phosphoric acid, monohydrogen phosphate, dihydrogen phosphate, sulfuric acid, hydrogen sulfate, hydroiodic acid, phosphorous acid, and the like; and an organic acid salt, wherein the organic acid includes, for example, acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, tartaric acid, and methanesulfonic acid, and the like; and salts of amino acid (such as arginine and the like), and a salt of an organic acid such as glucuronic acid and the like. Certain specific compounds of the present disclosure contain both basic and acidic functional groups, thus can be converted to any base or acid addition salt.

The pharmaceutically acceptable salt of the present disclosure can be prepared from the parent compound that contains an acidic or basic moiety by a conventional chemical method. Generally, such salt can be prepared by reacting the free acid or base form of the compound with a stoichiometric amount of an appropriate base or acid in water or an organic solvent or a mixture thereof.

The compounds of the present disclosure may exist in specific geometric or stereoisomeric forms. The present disclosure contemplates all such compounds, including cis and trans isomers, (−)- and (+)-enantiomers, (R)- and (S)-enantiomers, diastereoisomers, (D)-isomers, (L)-isomers, and racemic and other mixtures thereof, such as enantiomers or diastereomer enriched mixtures, all of which are within the scope of the present disclosure. Additional asymmetric carbon atoms may be present in substituents such as alkyl. All these isomers and their mixtures are encompassed within the scope of the present disclosure.

Unless otherwise specified, the term “enantiomer” or “optical isomer” refers to stereoisomers that are mirror images of each other.

Unless otherwise specified, the term “cis-trans isomer” or “geometric isomer” is caused by the inability to rotate freely of double bonds or single bonds of ring-forming carbon atoms.

Unless otherwise specified, the term “diastereomer” refers to a stereoisomer in which a molecule has two or more chiral centers and the relationship between the molecules is not mirror images.

Unless otherwise specified, “(+)” refers to dextrorotation, “(−)” refers to levorotation, and “(+)” refers to racemic.

Unless otherwise specified, the absolute configuration of a stereogenic center is represented by a wedged solid bond ( [symbol] ) and a wedged dashed bond ( [symbol] ), and the relative configuration of a stereogenic center is represented by a straight solid bond ( [symbol] ) and a straight dashed bond ( [symbol] ), a wave line ( [symbol] ) is used to represent a wedged solid bond ( [symbol] ) or a wedged dashed bond ( [symbol] ), or the wave line ( [symbol] ) is used to represent a straight solid bond ( [symbol] ) and a straight dashed bond ( [symbol] ). Unless otherwise specified, the term “tautomer” or “tautomeric form” means that at room temperature, the isomers of different functional groups are in dynamic equilibrium and can be transformed into each other quickly. If tautomers possibly exist (such as in solution), the chemical equilibrium of tautomers can be reached. For example, proton tautomer (also called prototropic tautomer) includes interconversion through proton migration, such as keto-enol isomerization and imine-enamine isomerization. Valence tautomer includes some recombination of bonding electrons for mutual transformation. A specific example of keto-enol tautomerization is the tautomerism between two tautomers of pentane-2,4-dione and 4-hydroxypent-3-en-2-one.

Unless otherwise specified, the terms “enriched in one isomer”, “enriched in isomers”, “enriched in one enantiomer” or “enriched in enantiomers” refer to the content of one of the isomers or enantiomers is less than 100%, and the content of the isomer or enantiomer is greater than or equal to 60%, or greater than or equal to 70%, or greater than or equal to 80%, or greater than or equal to 90%, or greater than or equal to 95%, or greater than or equal to 96%, or greater than or equal to 97%, or greater than or equal to 98%, or greater than or equal to 99%, or greater than or equal to 99.5%, or greater than or equal to 99.6%, or greater than or equal to 99.7%, or greater than or equal to 99.8%, or greater than or equal to 99.9%.

Unless otherwise specified, the term “isomer excess” or “enantiomeric excess” refers to the difference between the relative percentages of two isomers or two enantiomers. For example, if the content of one isomer or enantiomer is 90%, and the content of the other isomer or enantiomer is 10%, the isomer or enantiomer excess (ee value) is 80%.

Optically active (R)- and (S)-isomer, as well as (I))- and (L)-isomer can be prepared using chiral synthesis or chiral reagents or other conventional techniques. If one kind of enantiomer of certain compound of the present disclosure is to be obtained, the pure desired enantiomer can be obtained by asymmetric synthesis or derivative action of chiral auxiliary followed by separating the resulting diastereomeric mixture and cleaving the auxiliary group. Alternatively, when the molecule contains a basic functional group (such as amino) or an acidic functional group (such as carboxyl), the compound reacts with an appropriate optically active acid or base to form a salt of the diastereoisomer which is then subjected to diastereomeric resolution through the conventional method in the art to give the pure enantiomer. In addition, the enantiomer and the diastereoisomer are generally isolated through chromatography which uses a chiral stationary phase and optionally combines with chemical derivative method (such as carbamate generated from amine).

The compound of the present disclosure may contain an unnatural proportion of atomic isotope at one or more than one atom(s) that constitute the compound. For example, the compound can be radiolabeled with a radioactive isotope, such as tritium (³H), iodine-125 (¹²⁵I), or C-14 (¹⁴C). For another example, deuterated drugs can be formed by replacing hydrogen with heavy hydrogen, the bond formed by deuterium and carbon is stronger than that of ordinary hydrogen and carbon, compared with non-deuterated drugs, deuterated drugs have the advantages of reduced toxic and side effects, increased drug stability, enhanced efficacy, extended biological half-life of drugs, etc. All isotopic variations of the compound of the present disclosure, whether radioactive or not, are encompassed within the scope of the present disclosure.

The term “optional” or “optionally” means that the subsequently described event or circumstance may, but does not necessarily, occur, and the description includes instances where the event or circumstance occurs and instances where it does not.

The term “substituted” means one or more than one hydrogen atom(s) on a specific atom are substituted with the substituent, including deuterium and hydrogen variables, as long as the valence of the specific atom is normal and the substituted compound is stable. When the substituent is an oxygen (i.e., ═O), it means two hydrogen atoms are substituted. Positions on an aromatic ring cannot be substituted with a ketone. The term “optionally substituted” means an atom can be substituted with a substituent or not, unless otherwise specified, the type and number of the substituent may be arbitrary as long as being chemically achievable.

When any variable (such as R) occurs in the constitution or structure of the compound more than once, the definition of the variable at each occurrence is independent. Thus, for example, if a group is substituted by 0 to 2R, the group can be optionally substituted by up to two R, wherein the definition of R at each occurrence is independent. Moreover, a combination of the substituent and/or the variant thereof is allowed only when the combination results in a stable compound.

When the number of a linking group is 0, such as —(CRR)₀—, it means that the linking group is a single bond.

When one of the variables is selected from a single bond, it means that the two groups linked by the single bond are connected directly. For example, when L in A-L-Z represents a single bond, the structure of A-L-Z is actually A-Z.

When a substituent is vacant, it means that the substituent is absent, for example, when X is vacant in A-X, the structure of A-X is actually A. When the enumerative substituent does not indicate by which atom it is linked to the group to be substituted, such substituent can be bonded by any atom thereof. For example, when pyridyl acts as a substituent, it can be linked to the group to be substituted by any carbon atom on the pyridine ring.

When the enumerative linking group does not indicate the direction for linking, the direction for linking is arbitrary, for example, the linking group L contained in

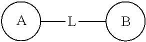

is -M-W-, then -M-W- can link ring A and ring B to form

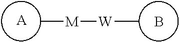

in the direction same as left-to-right reading order, and form

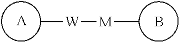

in the direction contrary to left-to-right reading order. A combination of the linking groups, substituents, and/or variables thereof is allowed only when such combination can result in a stable compound.

Unless otherwise specified, when a group has one or more linkable sites, any one or more sites of the group can be linked to other groups through chemical bonds. When the linking site of the chemical bond is not positioned, and there is an H atom at the linkable site, then the number of H atoms at the site will decrease correspondingly with the number of chemical bonds linking thereto so as to meet the corresponding valence. The chemical bond between the site and other groups can be represented by a straight solid bond ( [symbol] ), a straight dashed bond ( [symbol] ) or a wavy line ( [symbol] ). For example, the straight solid bond in —OCH₃ means that it is linked to other groups through the oxygen atom in the group; the straight dashed bond in [symbol] means that it is linked to other groups through the two ends of the nitrogen atom in the group; the wave lines in

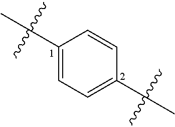 [SMILES: *c1ccc(*)cc1]

means that the phenyl group is linked to other groups through carbon atoms at position 1 and position 2;

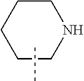

means that it can be linked to other groups through any linkable sites on the piperidinyl by one chemical bond, including at least four types of linkage, including

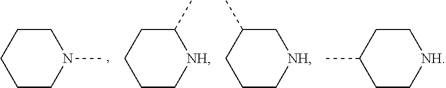

Even though the H atom is drawn on the —N—,

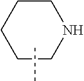

still includes the linkage of

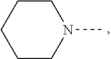

merely when one chemical bond was connected, the H of this site will be reduced by one to the corresponding monovalent piperidinyl.

Unless otherwise specified, when a double bond structure, such as carbon-carbon double bond, carbon-nitrogen double bond, and nitrogen-nitrogen double bond, exists in the compound, and each of the atoms on the double bond is connected to two different substituents (including the condition where a double bond contains a nitrogen atom, the lone pair of electrons attached on the nitrogen atom is regarded as a substituent connected), if the atom on the double bond in the compound is connected to its substituent by

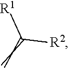

this refers to the (Z)-isomer, (E)-isomer or a mixture of two isomers of the compound.

Unless otherwise specified, the term “C₁₋₃ alkyl” refers to a linear or branched saturated hydrocarbon group consisting of 1 to 3 carbon atoms. The C₁₋₃ alkyl includes C₁₋₂ and C₂₋₃ alkyl, etc.; it can be monovalent (such as methyl), divalent (such as methylene), or multivalent (such as methine). Examples of C₁₋₃ alkyl include, but are not limited to, methyl (Me), ethyl (Et), propyl (including n-propyl and isopropyl), etc.

Unless otherwise specified, the term “C₁₋₃ alkoxy” refers to an alkyl group consisting of 1 to 3 carbon atoms that are connected to the rest of the molecule through an oxygen atom. The C₁₋₃ alkoxy includes C₁₋₂, C₂₋₃, C₃, and C₂ alkoxy, etc. Examples of C₁₋₃ alkoxy include, but are not limited to, methoxy, ethoxy, propoxy (including n-propoxy and isopropoxy), etc.

Unless otherwise specified, the term “C₃₋₇ cycloalkyl” refers to a saturated cyclic hydrocarbon group consisting of 3 to 7 carbon atoms, which includes monocyclic and bicyclic systems, wherein the bicyclic system includes a spiro ring, a fused ring, and a bridged ring. The C₃₋₇ cycloalkyl includes C₃₋₆, C₃₋₅, C₄₋₇, C₄₋₆, C₄₋₅, C₅₋₇, or C₅₋₆ cycloalkyl, etc.; it can be monovalent, divalent, or multivalent. Examples of C₃₋₇ cycloalkyl include, but are not limited to, cyclopropyl, cyclobutyl, cyclopentyl, cyclohexyl, cycloheptyl, norbornyl, spiroheptyl, etc.

The term “leaving group” refers to a functional group or atom which can be replaced by another functional group or atom through a substitution reaction (such as affinity substitution reaction). For example, representative leaving groups include trifluoromethanesulfonate; chlorine, bromine, and iodine; sulfonate group, such as methanesulfonate, toluenesulfonate, p-bromobenzenesulfonate, p-toluenesulfonate, etc; acyloxy, such as acetoxy, trifluoroacetoxy, etc.

The term “protecting group” includes, but is not limited to, “amino protecting group”, “hydroxyl protecting group” or “mercapto protecting group”. The term “amino protecting group” refers to a protecting group suitable for preventing the side reactions occurring at the nitrogen of an amino. Representative amino protecting groups include, but are not limited to: formyl; acyl, such as alkanoyl (e.g., acetyl, trichloroacetyl, or trifluoroacetyl); alkoxycarbonyl, such as tert-butoxycarbonyl (Boc); arylmethoxycarbonyl, such as benzyloxycarbonyl (Cbz) and 9-fluorenylmethoxycarbonyl (Fmoc); arylmethyl, such as benzyl (Bn), trityl (Tr), 1,1-bis-(4′-methoxyphenyl)methyl; silyl, such as trimethylsilyl (TMS) and tert-butyldimethylsilyl (TBS), etc. The term “hydroxyl protecting group” refers to a protecting group suitable for preventing the side reactions of hydroxyl. Representative hydroxyl protecting groups include, but are not limited to: alkyl, such as methyl, ethyl, and tert-butyl; acyl, such as alkanoyl (e.g., acetyl); arylmethyl, such as benzyl (Bn), p-methoxybenzyl (PMB), 9-fluorenylmethyl (Fm), and diphenylmethyl (benzhydryl, DPM); silyl, such as trimethylsilyl (TMS) and tert-butyldimethylsilyl (TBS), etc.

The compounds of the present disclosure can be prepared by a variety of synthetic methods known to those skilled in the art, including the specific embodiments listed below, the embodiments formed by their combination with other chemical synthesis methods, and equivalent alternatives known to those skilled in the art, preferred embodiments include but are not limited to the examples of the present disclosure.

The structure of the compounds of the present disclosure can be confirmed by conventional methods known to those skilled in the art, and if the disclosure involves an absolute configuration of a compound, then the absolute configuration can be confirmed by means of conventional techniques in the art. For example, in the case of single crystal X-ray diffraction (SXRD), the absolute configuration can be confirmed by collecting diffraction intensity data from the cultured single crystal using a Bruker D8 venture diffractometer with CuKa radiation as the light source and scanning mode: p/@ scan, and after collecting the relevant data, the crystal structure can be further analyzed by the direct method (Shelxs97).

The compounds of the present disclosure are named according to the conventional naming principles in the art or by ChemDraw® software, and the commercially available compounds use the supplier catalog names.

### DETAILED DESCRIPTION OF THE PREFERRED EMBODIMENT

The present disclosure is described in detail by the examples below, but it does not mean that there are any adverse restrictions on the present disclosure. The compounds of the present disclosure can be prepared by a variety of synthetic methods known to those skilled in the art, including the specific embodiments listed below, the embodiments formed by their combination with other chemical synthesis methods, and equivalent alternatives known to those skilled in the art, preferred embodiments include but are not limited to the examples of the present disclosure. For one skilled in the art, it is obvious to make various modifications and improvements to the embodiments of the present disclosure without departing from the spirit and scope of the present disclosure.

### Example 1

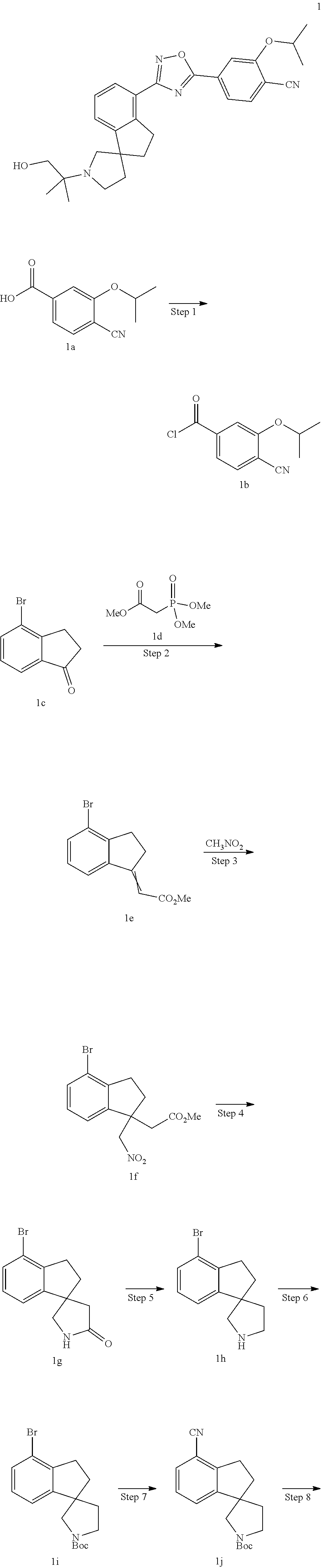

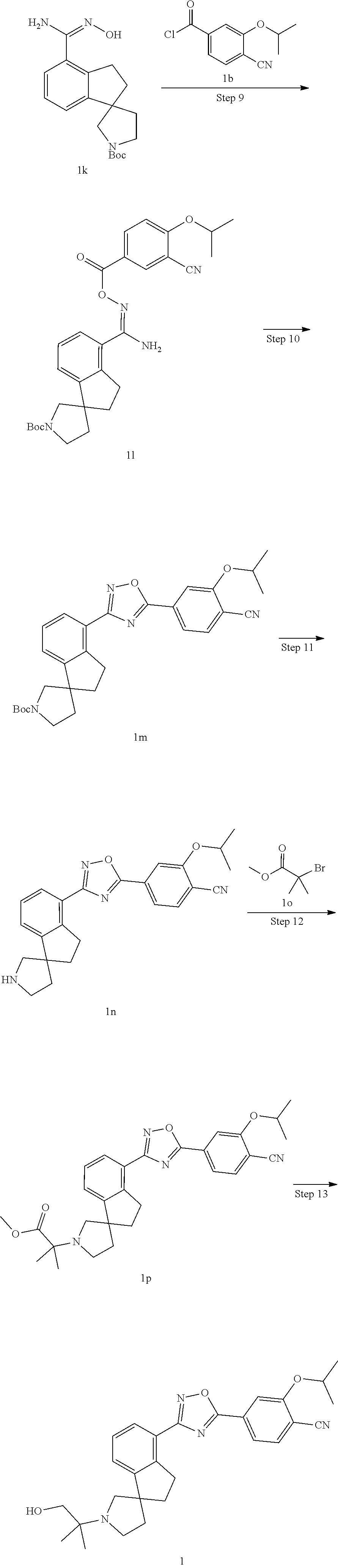

### Step 1

Compound 1a (2 g, 9.75 mmol) was dissolved in dichloromethane (20 mL). To the system was sequentially added oxalyl chloride (3.71 g, 29.24 mmol) and N,N-dimethylformamide (71.24 mg, 974.61 μmol) at 0° C. The reaction mixture was stirred at 20° ° C. for 0.5 hours. The reaction mixture was concentrated under reduced pressure to remove the solvent, and the resulting crude product 1b was directly used in a later step.

### Step 2

Compound 1d (280.43 g, 1.54 mol) was dissolved in N,N-dimethylformamide (100 mL). To the system was added potassium tert-butoxide (166.15 g, 1.48 mol) in batches at 25° ° C. The reaction mixture was stirred at 25ºC for 3 hours, then slowly added with compound 1c (250 g, 1.18 mol) in batches, and stirred at 25° C. for 13 hours. Other parallel batches (comprising an addition of 840 g of 1d) were combined for further processing. To the reaction mixture was added water (1600 mL). The reaction mixture was extracted with ethyl acetate (6 L×5). The combined organic phases were washed with saturated brine (1.6 L×1), dried over anhydrous sodium sulfate, filtered, and concentrated to obtain a crude product. The crude product was purified by neutral alumina column chromatography (petroleum ether/ethyl acetate, 2/1 to 1/1, V/V) to obtain compound 1e. MS-ESI calculated for [M+H]267 and 269, and found 267 and 269.

### Step 3

Compound 1e (767 g, 2.87 mol) was dissolved in dimethyl sulfoxide (3000 mL). To the system was added cesium carbonate (748.44 g, 2.30 mol) at 25° C. The reaction mixture was heated to 70° C., and to the system was slowly added dropwise nitromethane (525.81 g, 8.61 mol). The reaction mixture was stirred at 70° C. for 12 hours. Other parallel batches (comprising an addition of 767 g of 1e) were combined for further processing. The reaction mixture was added with water (2400 mL), and extracted with ethyl acetate (10 L×1, 5 L×1). The combined organic phases were washed with saturated brine (5 L×2), dried over anhydrous sodium sulfate (2 kg), filtered, and concentrated to obtain a crude product. The crude product was purified by neutral alumina column chromatography (petroleum ether/ethyl acetate, 1/1, V/V) to obtain compound 1f.

### Step 4

Compound 1f (725 g, 2.21 mol) was dissolved in ethanol (2175 mL) and water (725 mL). To the system was added ammonium chloride (354.54 g, 6.63 mol) at 25° C., and the reaction mixture was heated to 80° C. To the system was then slowly added iron powder (370.14 g, 6.63 mol) in batches, and the reaction mixture was stirred at 80° C. for 12 hours. Other parallel batches (comprising an addition of 725 g of 1f) were combined for further processing. The reaction mixture was filtered, and the filter cake was sequentially rinsed with ethyl acetate (8 L) and water (4 L). The filtrate was concentrated to remove some of the solvent, then added with water (4 L), and extracted with ethyl acetate (4 L×2). The combined organic phases were washed with saturated brine (3 L×2), dried over anhydrous sodium sulfate (2 kg), filtered, and concentrated to obtain a crude product. At 25° C., the crude product was slurried with a mixed solvent (n-heptane/ethyl acetate, 1/1, V/V), stirred for 12 hours, filtered, and dried to obtain compound 1g. MS-ESI calculated for [M+H]⁺ 266 and 268, and found 266 and 268.

### Step 5

Compound 1g (50 g, 178.06 mmol) was dissolved in tetrahydrofuran (350 mL). To the system was added dropwise a solution of borane-dimethylsulfide (10 M, 35.61 mL) at 20° C. The reaction mixture was then heated to 70° C., and stirred at 70° C. for 12 hours. Other parallel batches (comprising an addition of 50 g of 1g) were combined for further processing. The reaction mixture was poured into hydrochloric acid aqueous solution (1 M, 400 mL) at 80° C., and extracted with ethyl acetate (200 mL×3). The resulting aqueous phase was added with saturated sodium carbonate aqueous solution to adjust the pH to about 9, and extracted with dichloromethane (300 mL×3). The combined organic phases were dried over anhydrous sodium sulfate (100 g), filtered, and concentrated to obtain a crude product. The crude product 1h was directly used in the next step without further purification. MS-ESI calculated for [M+H]⁺ 252 and 254, and found 252 and 254.

### Step 6

Compound 1h (22 g, 87.25 mmol) was dissolved in dichloromethane (130 mL). To the system was added triethylamine (21.19 g, 209.40 mmol) and di-tert-butyl dicarbonate (22.85 g, 104.70 mmol). The reaction mixture was stirred at 20ºC for 12 hours. Other parallel batches (comprising an addition of 22 g of 1g) were combined for further processing. To the system was added water (800 mL). The reaction mixture was extracted with dichloromethane (500 mL×2). The combined organic phases were dried over anhydrous sodium sulfate (100 g), filtered, and concentrated to obtain a crude product. The crude product was purified by silica gel column chromatography (petroleum ether/ethyl acetate, 100/1 to 10/1, V/V) to obtain compound 1i. MS-ESI calculated for [M−/Bu+H]⁺ 296 and 298, and found 296 and 298.

### Step 7

To N,N-dimethylformamide (400 mL) was sequentially added compound 1i (42.9 g, 121.78 mmol), tris(dibenzylideneacetone) (4.46 g, 4.87 mmol), 2-dicyclohexylphosphino-2′,4′,6′-triisopropylbiphenyl (4.64 g, 9.74 mmol), and zinc cyanide. The reaction system was replaced with nitrogen, stirred at 90° ° C. for 12 hours, added with water (1000 mL), and filtered. The filter cake was washed with ethyl acetate (200 mL×2). The aqueous phase was extracted with ethyl acetate (300 mL×2). The combined organic phases were dried over anhydrous sodium sulfate, filtered, and concentrated to obtain a crude product. The crude product was purified by silica gel column chromatography (petroleum ether/ethyl acetate, 50/1 to 10/1, V/V) to obtain compound 1j. MS-ESI calculated for [M−/Bu+H]⁺ 243, and found 243.

### Step 8

Compound 1j (12.5 g, 41.89 mmol) was dissolved in ethanol (120 mL). To the system was sequentially added hydroxylamine hydrochloride (4.64 g, 9.74 mmol) and triethylamine (4.64 g, 9.74 mmol). The reaction mixture was stirred at 80° C. for 5 hours. The reaction mixture was concentrated, and then water (200 mL) was added thereto. The aqueous phase was extracted with ethyl acetate (150 mL×2). The combined organic phases were washed with water (100 mL×3), dried over anhydrous sodium sulfate, filtered, and concentrated to obtain a crude product. The crude product 1k was directly used in the next step without further purification. MS-ESI calculated for [M-Bu+H]⁺ 276, and found 276.

### Step 9

Compound 1k (2.7 g, 7.14 mmol) and compound 1b (2.08 g, 9.28 mmol) were dissolved in dichloromethane (30 mL). To the system was added dropwise N,N-diisopropylethylamine (2.77 g, 21.41 mmol). The reaction mixture was stirred at 20° ° C.for 12 hours. The reaction mixture was concentrated, and then water (80 mL) was added thereto. The aqueous phase was extracted with dichloromethane (40 mL×2). The combined organic phases were dried over anhydrous sodium sulfate (10 g), filtered, and concentrated to obtain a crude product. The crude product 1I was directly used in the next step without further purification. MS-ESI calculated for [M−/Bu+H]⁺ 463, and found 463.

### Step 10

Compound 1I (5 g, 9.64 mmol) was dissolved in acetonitrile (50 ml). To the system was added sodium hydroxide (1.5 g, 38.57 mmol). The reaction mixture was stirred at 27° ° C. for 12 hours. To the system was added water (80 mL). The aqueous phase was extracted with ethyl acetate (40 mL×2). The combined organic phases were dried over anhydrous sodium sulfate (5 g), filtered, and concentrated to obtain a crude product. The crude product was purified by silica gel column chromatography (petroleum ether/ethyl acetate, 3/1, V/V) to obtain compound 1m. MS-ESI calculated for [M−/Bu+H]⁺ 445, and found 445.

### Step 11

Compound 1m (2.1 g, 4.20 mmol) was dissolved in ethyl acetate (20 mL). To the system was added a solution of hydrogen chloride in ethyl acetate (4 M, 20 mL). The reaction mixture was stirred at 20° ° C. for 0.5 hours. The reaction mixture was concentrated under reduced pressure to obtain a crude product. The crude product, hydrochloride of compound 1n, was directly used in the next step without further purification. MS-ESI calculated for [M+H]⁺ 401, and found 401.

### Step 12

The hydrochloride of compound 1n (1 g, 2.29 mmol) was dissolved in acetonitrile (15 mL). To the system was sequentially added potassium iodide (189.96 mg, 1.14 mmol), potassium carbonate (948.93 mg, 6.87 mmol), and compound 10 (621.47 mg, 3.43 mmol). The reaction mixture was stirred at 85ºC for 12 hours. To the system was added water (30 mL). The aqueous phase was extracted with ethyl acetate (20 mL×2). The combined organic phases were dried over anhydrous sodium sulfate (5 g), filtered, and concentrated to obtain a crude product. The crude product was purified by silica gel column chromatography (petroleum ether/ethyl acetate, 5/1 to 3/1, V/V) to obtain compound 1p. MS-ESI calculated for [M+H]⁺ 501, and found 501.

### Step 13

Compound 1p (810 mg, 1.62 mmol) was dissolved in tetrahydrofuran (10 mL). To the system was added lithium borohydride (110 mg, 5.05 mmol). The reaction mixture was stirred at 20° C. for 0.5 hours, then heated to 70° C., and stirred continuously for 12 hours. To the system was added hydrochloric acid aqueous solution (1 N, 30 mL). The aqueous phase was extracted with ethyl acetate (30 mL×2). The combined organic phases were dried over anhydrous sodium sulfate (10 g), filtered, and concentrated to obtain a crude product. The crude product was purified by preparative HPLC (chromatographic column: Waters Xbridge C18 150× 50 mm×10 um; mobile phase: 10 mmol/L ammonium bicarbonate aqueous solution-acetonitrile; gradient: acetonitrile from 62% to 92%, 10 minutes) to obtain compound 1. MS-ESI calculated for [M+H]⁺ 473, and found 473. ¹H NMR (400 MHZ, CD₃OD) δ=8.47-8.41 (m, 2H), 8.01 (d, J=7.6 Hz, 1H), 7.54 (d, J=7.4 Hz, 1H), 7.48-7.37 (m, 2H), 5.00-4.94 (m, 1H), 3.47 (s, 2H), 3.31-3.27 (m, 2H), 3.11-3.03 (m, 1H), 3.03-2.95 (m, 3H), 2.30-2.22 (m, 1H), 2.21-2.13 (m, 1H), 2.11-1.96 (m, 2H), 1.48 (d, J=6.1 Hz, 6H), 1.14 (s, 6H).

Bioactivity Evaluations

### Test example 1: Evaluating the in vitro agonist activity of the compound of the present disclosure on S1PR1

Experimental purpose: To assess the agonist activity of the compound on S1PR1

I. Cell Treatment
1. The PathHunter cell line was thawed according to standard procedures.
2. The cells were seeded in a 384-well plate with a volume of 20 μL, and incubated at 37° C. for an appropriate duration.

II. Agonist
1. For the agonist evaluation, the cells were co-cultured with the test samples to induce a reaction.
2. The test stock solution was diluted 5-fold using the buffer.
3. 5 μL of the 5-fold diluted solution was added to the cells, and incubated at 37° C. for 90 to 180 minutes. The vehicle concentration was maintained at 1%.

III. Signal Detection
1. Either 12.5 μL or 15 μL of the PathHunter detection reagent (at a 50% v/v ratio) was added each time, followed by a 1-hour incubation at room temperature to allow for detection signal generation.
2. Chemiluminescence signal detection was conducted using a PerkinElmer Envision™ microplate reader.

IV. Data Analysis
1. The activity of the compound was analyzed using the CBIS suite for data analysis (ChemInnovation, CA).
2. Formula for calculation:

% Activity=100%× (Average sample RLU—Average vehicle RLU)/(Average maximum control ligand RLU—Average vehicle RLU)

The results of the experiment are shown in Table 1:

TABLE 1
Results of the S1PR1 agonist activity test

| Test Sample | S1PR1 Agonist Activity, Emax |
|---|---|
| Compound 1 | 0.657 nM, 110% |

Conclusion: The compound of the present disclosure demonstrates significant and even unexpected S1PR1 agonist activity.

What is claimed is:

1. A compound of formula (I) or a pharmaceutically acceptable salt thereof, 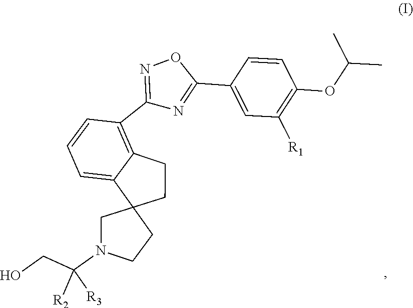
wherein
R₁ is selected from H, F, Cl, Br, I, OH, NH₂, CN, C₁₋₃ alkyl, and C₁₋₃ alkoxy, wherein the C₁₋₃ alkyl and C₁₋₃ alkoxy are each independently and optionally substituted by 1, 2, or 3 Rₐ;
R₂ is selected from H, C₁₋₃ alkyl, and C₁₋₃ alkoxy, wherein the C₁₋₃ alkyl and C₁₋₃ alkoxy are each independently and optionally substituted by 1, 2, or 3 R_{b};
R₃ is selected from C₁₋₃ alkyl and C₁₋₃ alkoxy, wherein the C₁₋₃ alkyl and C₁₋₃ alkoxy are each independently and optionally substituted by 1, 2, or 3 R_{c};
or, R₂ and R₃ together with the carbon atom to which they are attached form a C₃₋₇ cycloalkyl ring;
Rₐ, R_{b}, and Re are each independently selected from F, Cl, Br, I, OH, NH₂, CN, and COOH.

2. The compound or the pharmaceutically acceptable salt thereof according to claim 1, wherein Rₐ, R_{b}, and Re are each independently selected from F, Cl, and Br.

3. The compound or the pharmaceutically acceptable salt thereof according to claim 1 or 2, wherein R₁ is selected from H, F, Cl, Br, CN, —CH₃, and 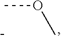
wherein the —CH₃ and —OCH₃ are each independently and optionally substituted by 1, 2, or 3 Rₐ.

4. The compound or the pharmaceutically acceptable salt thereof according to claim 3, wherein each R₁ is independently selected from H, F, Cl, Br, CN, —CH_{3,}

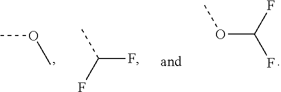

5. The compound or the pharmaceutically acceptable salt thereof according to claim 1 or 2, wherein R₂ is selected from H, —CH₃, —CH₂CH₃, 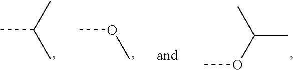
and the —CH₃, —CH₂CH₃, 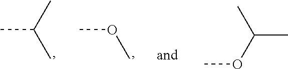
are each independently and optionally substituted by 1, 2, or 3 R_{b}.

6. The compound or the pharmaceutically acceptable salt thereof according to claim 5, wherein R₂ is selected from H, —CH₃, —CH₂CH₃,

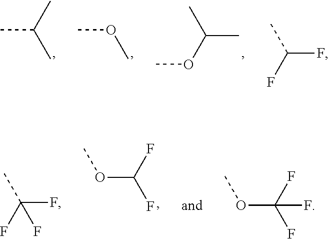

7. The compound or the pharmaceutically acceptable salt thereof according to claim 1 or 2, wherein R₃ is selected from —CH₃, —CH₂CH₃, 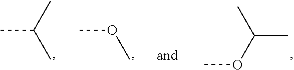
wherein the —CH₃, —CH₂CH₃, 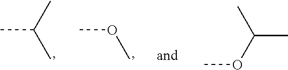
are each independently and optionally selected from 1, 2, or 3 R_{c}.

8. The compound or the pharmaceutically acceptable salt thereof according to claim 7, wherein R₃ is selected from —CH₃, —CH₂CH_{3,}

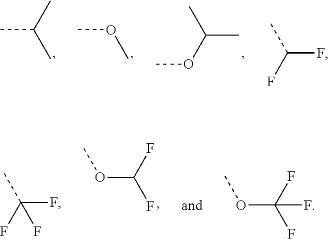

9. The compound or the pharmaceutically acceptable salt thereof according to claim 1 or 2, wherein R₂ and R₃ together with the carbon atom to which they are attached form

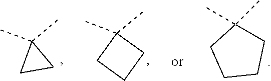

10. The compound or the pharmaceutically acceptable salt thereof according to any one of claim 6, 8, or 9, wherein the structural moiety 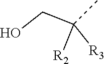
is selected from

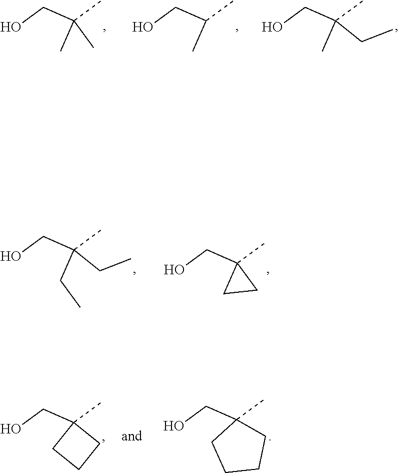

11. The compound or the pharmaceutically acceptable salt thereof according to claim 1 or 2, wherein the structural moiety 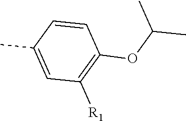
is selected from

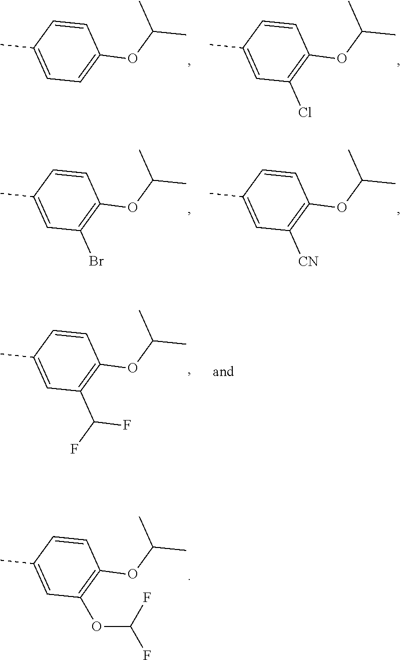

12. A compound of the following formula or a pharmaceutically acceptable salt thereof,

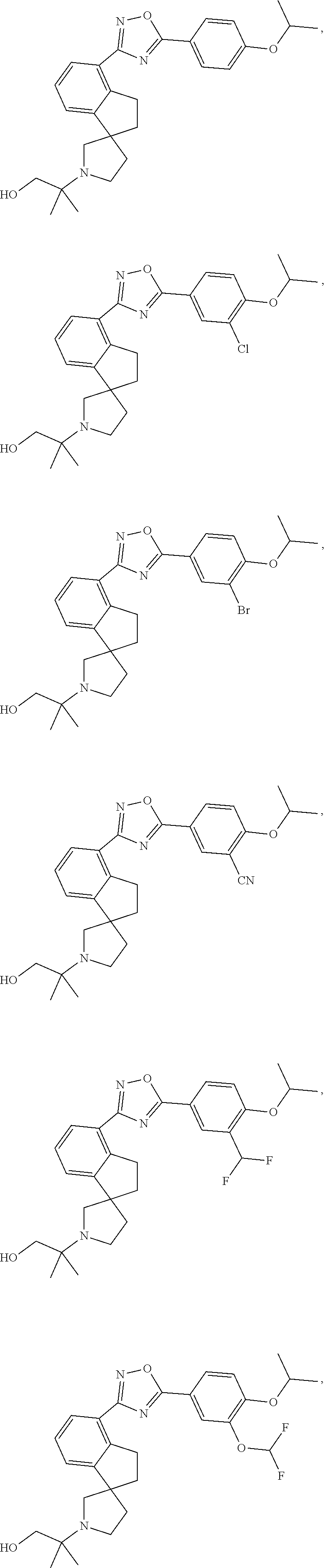 [SMILES: CC(C)Oc1ccc(-c2nc(-c3cccc4c3CCC43CCN(C(C)(C)CO)C3)no2)cc1 ; CC(C)Oc1ccc(-c2nc(-c3cccc4c3CCC43CCN(C(C)(C)CO)C3)no2)cc1Cl ; CC(C)Oc1ccc(-c2nc(-c3cccc4c3CCC43CCN(C(C)(C)CO)C3)no2)cc1Br ; CC(C)Oc1ccc(-c2nc(-c3cccc4c3CCC43CCN(C(C)(C)CO)C3)no2)cc1C#N ; CC(C)Oc1ccc(-c2nc(-c3cccc4c3CCC43CCN(C(C)(C)CO)C3)no2)cc1C(F)F ; CC(C)Oc1ccc(-c2nc(-c3cccc4c3CCC43CCN(C(C)(C)CO)C3)no2)cc1OC(F)F]

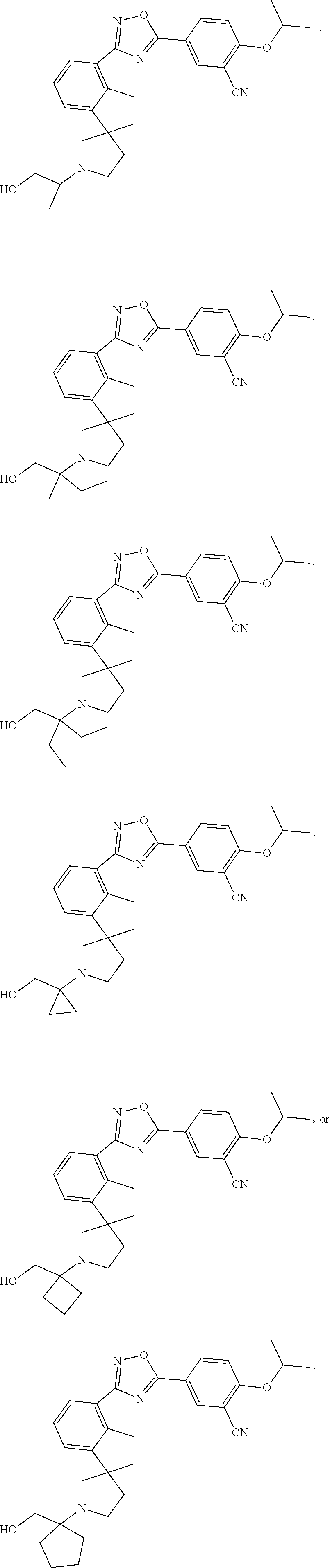 [SMILES: CC(C)Oc1ccc(-c2nc(-c3cccc4c3CCC43CCN(C(C)CO)C3)no2)cc1C#N ; CCC(C)(CO)N1CCC2(CCc3c(-c4noc(-c5ccc(OC(C)C)c(C#N)c5)n4)cccc32)C1 ; CCC(CC)(CO)N1CCC2(CCc3c(-c4noc(-c5ccc(OC(C)C)c(C#N)c5)n4)cccc32)C1 ; CC(C)Oc1ccc(-c2nc(-c3cccc4c3CCC43CCN(C4(CO)CC4)C3)no2)cc1C#N ; CC(C)Oc1ccc(-c2nc(-c3cccc4c3CCC43CCN(C4(CO)CCC4)C3)no2)cc1C#N ; CC(C)Oc1ccc(-c2nc(-c3cccc4c3CCC43CCN(C4(CO)CCCC4)C3)no2)cc1C#N]

13. The compound or the pharmaceutically acceptable salt thereof according to claim 12,

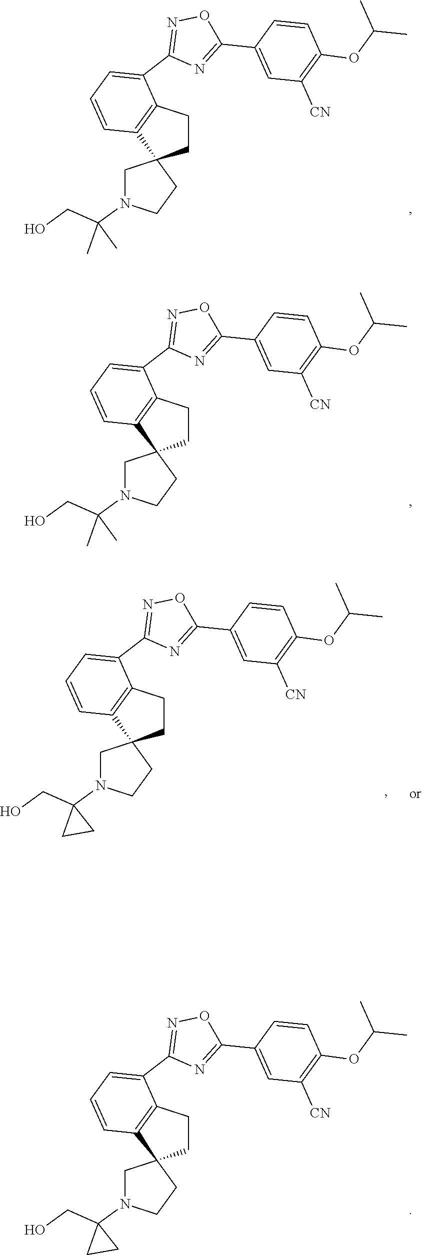 [SMILES: CC(C)Oc1ccc(-c2nc(-c3cccc4c3CC[C@]43CCN(C(C)(C)CO)C3)no2)cc1C#N ; CC(C)Oc1ccc(-c2nc(-c3cccc4c3CC[C@@]43CCN(C(C)(C)CO)C3)no2)cc1C#N ; CC(C)Oc1ccc(-c2nc(-c3cccc4c3CC[C@]43CCN(C4(CO)CC4)C3)no2)cc1C#N ; CC(C)Oc1ccc(-c2nc(-c3cccc4c3CC[C@@]43CCN(C4(CO)CC4)C3)no2)cc1C#N]

14. A use of the compound or the pharmaceutically acceptable salt thereof according to any one of claims 1 to 13 in the manufacture of a medicament for treating S1PR1 receptor.
